(19) United States
(12) Patent Application Publication
(10) Pub. No.: US 2024/0158253 A1
(43) Pub. Date: May 16, 2024

(54) Optically Stable Transparent Zinc Oxide for Dental Applications

(71) Applicant: Den-Mat Holdings, LLC, Lompoc, CA (US)

(72) Inventors: Alvin Kobashigawa, Mission Viejo, CA (US); Daehwan Shin, Naperville, IL (US)

(73) Assignee: Den-Mat Holdings, LLC, Lompoc, CA (US)

(21) Appl. No.: 17/985,783

(22) Filed: Nov. 11, 2022

(51) Int. Cl. C01G 9/02; A61K 8/27

(52) U.S. Cl. CPC C01G 9/02; A61K 8/27

(57) ABSTRACT

A method of stabilizing nanoparticle zinc oxide and the incorporation of nanopoarticle zinc oxide into a wide range of dental compositions. The use of nanoparticle zinc oxide allows for greater concentrations of zinc oxide to be incorporated into dental compositions while maintaining translucency and increased antimicrobial activity compared to equivalent concentrations of bulk zinc oxide.

### CROSS-REFERENCE TO RELATED APPLICATIONS

This application is a continuation of U.S. provisional patent application 63/279,011 filed on Nov. 12, 2021, entitled, “Optically Stable Transparent Zinc Oxide For Dental Applications.”

### FIELD OF THE INVENTION

The present invention relates to improvements in tooth treatment compositions. Specifically, this invention relates to the use of transparent zinc oxide to improve dental restorative materials by enhancing the beneficial qualities of zinc oxide in dental restoratives while minimizing the negative aesthetic results of traditional zinc oxide formulations in dental restoratives.

### BACKGROUND OF THE INVENTION

Zinc oxide has been used extensively in dental restorative materials. It is used in cements to lute in restorations and in bases for lining cavity preparations. Some examples of dental cements that utilize zinc oxide includes cements based on zinc phosphate, polycarboxylate, zinc oxide and eugenol, as well as some glass ionomer cements. When used in these cements, there is an acid-base reaction, involving an inorganic or organic acid and zinc oxide particles resulting in the cement's setting action.

In zinc phosphate cements, the acid is phosphoric acid. In polycarboxylate cements, the acid is polyacrylic acid. And in zinc oxide and eugenol cements, the acidic material is eugenol. The main reason for the use of these materials is ease-of-use and clinical longevity. All these materials have an established history of long-term clinical performance.

The long-term clinical performance of zinc oxide is not just from the setting reaction, but also from of zinc oxide's antimicrobial activity. Zinc oxide is mildly antimicrobial and has a history of use in medications. Some well-known uses of zinc oxides includes: zinc oxide topical skin to protect against boils and carbuncles, calamine lotions to relieve skin irritations and in shampoos to protect against dandruff. Soluble zinc compounds are used in lozenges and nasal sprays to protect against colds.

Despite the benefits of zinc oxide in dental materials, it is not an esthetic material. It is very opaque and as a result makes restorations look “chalky white” in appearance. For this reason, the use of zinc oxide has been primarily limited to applications such as cements, bases and root canal fillings that do not require esthetics. For more esthetic applications, such as restoratives, zinc oxide cannot be used in high concentrations in esthetic restorative materials because of its opacity. As a results, these esthetic applications are not able to receive the full benefits of zinc oxide.

### BRIEF DESCRIPTION OF THE INVENTION

Recently, zinc oxide has been made available in transparent nanoparticle form. That means that the bulk particle size of many materials is reduced to the nanoparticle range (1-100 nanometers). The challenge of using zinc oxide in this transparent nanoparticle form is that the physical properties change dramatically. The reason for the drastic change in physical properties is due to the increase in surface area and quantum effects with reduced particle size. There is a critical particle size where the color, translucency, catalytic activity, strength etc. are changed dramatically. That means that simply substituting transparent nanoparticle zinc oxide into a dental restorative will not result a dental restorative with the same properties as standard sized zinc oxide.

For opaque zinc oxide particles, when the particle size is reduced to below 15 nm, the material becomes completely transparent. In this range, the particles are smaller than the critical wavelength of visible light and do not interact with light waves. Consequently, the particles become completely transparent. For example, in a zinc oxide based sunscreen, use of zinc oxide particles of this size would result in a transparent sunscreen which may be applied anywhere on the skin and is not unsightly in facial areas. That is in contrast to zinc oxide-based sunscreens which utilize a larger particle sized zinc oxide and have an opaque, chalky white appearance. The similar concept applies with dental restoratives—transparent zinc oxide nanoparticles may now be applied to esthetic restorative materials with the full benefits of its antimicrobial activity along with the setting activity.

This invention discusses how transparent zinc oxide nanoparticles, particularly in the critical range of 1-15 nm range, have been synthesized, stabilized and adapted for use in esthetic dental restorative materials while taking into account the variations in the chemical properties of zinc oxide in nanoparticle form.

The nanoparticles may be used as is or reacted with coupling agents similar to the cement setting reaction to keep the particle size below the critical 1-15 nm range. Reacting the zinc oxide nanoparticles stabilizes the zinc oxide particles and prevents the zinc oxide from growth and maintains the optical transparency throughout the restorative life. If the zinc oxide nanoparticles are not stabilized below this 15 nm range, continued particle size growth will cause the return of opacity. Not all nanoparticles are transparent. Particles must be formulated to be in the 1-15 nanometer range and must be discrete. They must also be reacted to prevent further growth in particle size, such as the method described in Example 2 below.

Use of nanoparticle zinc oxide is not limited to esthetic dental restoratives such as adhesives, high-performance restorative materials and esthetic cements. It can be extended to include all dental applications including liners, bases and any other application that would benefit from zinc oxide's long long-term clinical performance would be desirable. This invention's stable zinc oxide nanoparticles may also be used in other non-dental applications such as transparent ointments and sunscreens.

### DETAILED DESCRIPTION OF THE INVENTION

Transparent zinc oxide nanoparticles may be synthesized in controlled reactions that limit the particle size. One method dissolves zinc acetate in ethanol and then reacts the subsequent mixture with sodium hydroxide. Zinc oxide nanoparticles then formed, and the particle size is controlled by time. After 24 hours, the solution should still be transparent, at which butanediolmonoacrylate (BDMA) is added and the solvent ethanol is removed by rotary vacuum. The product is optically stable over time. Polymerization is accomplished by adding trimethylolpropanetriacrylate and photopolymerizing with lucerin TPO and UV light. When polymerized, the particles are locked into the matrix and are stable over time. The nanoparticles are chemically pure zinc oxide.

An alternate method for producing transparent zinc oxide nanoparticles for utilizes the same materials but uses isopropyl alcohol as the solvent instead of ethanol. The absorption wavelength is a function of particle size when the particles are small. The synthesis involves particle size growth at 65° C. with time and particle size is measured by the absorption wavelength with time. Eventually, the particle size exceeds the critical wavelength of visible light and the particles become opaque, as with bulk zinc oxide particles.

For dental applications, optically stable transparent zinc oxide particles were synthesized by two methods. First, the particles may be stabilized by polymerizing into a resin matrix as described above. Hydroxyethyl methacrylate (HEMA) was substituted for butanediolmonoacrylate in this method because HEMA a common monomer in dental formulations and may be incorporated into dental resins. Both monomers are similar in chemical structure, so similar behavior with respect to nanoparticle stability was expected. However, stability in HEMA was only about one week with opaque particles forming beyond this time. In order to keep the particles stable and prevent opaque particles from forming, it is necessary to polymerize the matrix and mill the material as an additive.

Long-term stable, transparent zinc oxide nanoparticles may also be obtained by reacting the nanoparticles with coupling agents, such as carboxylic acid methacrylates or phosphoric acid methacrylates. These coupling agents may react with zinc oxide surface similar to the reaction of polycarboxylate and zinc phosphate cements respectively, to stabilize the particle from further growth. The antimicrobial activity would be similar to polycarboxylate and zinc phosphate cements respectively. For strength and stability, which may be required for dental restorative materials, the methacrylate functionality is available to react with matrix resins.

Example 1 describes how long-term stable, transparent zinc oxide nanoparticles may be synthesized and stabilized with beta carboxyethyl acrylate (BCEA). BCEA is a preferable monomer because it is soluble in ethanol and the reacted product is stable in both ethanol and HEMA resin for extended periods. Other similar carboxylic acid methacrylate monomers may be used for this purpose. Examples are: maleic and succinic acid methacrylates, 4-Methacryloxyethyl trimellitic anhydride (4-META), Pyromellitic Acid Dimethacrylate (PMDM), Phthalic Acid Monomethacrylate (PAMM) and other acidic monomers typically used in dental adhesives.

### Example 1. Synthesis of Zinc Oxide Nanoparticles Stabilized with Beta Carboxyethyl Acrylate

1. Dissolve zinc acetate in ethanol. Add a stirring bar and mix on a hotplate at about 68° C. until dissolved.
2. Add of a solution containing ethanol and NaOH solution and stir for at least 30 mins. Let cool to room temperature and let stand at least 8 hours.
3. Dissolve beta carboxyethyl acrylate in ethanol. Add slowly to the nanoparticle solution to adjust the nanoparticle solution to pH 5.
4. Add hydroxyethylmethacrylate or triethyleneglycol dimethacrylate (Tegdma) and remove ethanol by rotary vacuum. The zinc oxide is stable in the resin.

Similar stable zinc oxide nanoparticles may be obtained by treating the nanoparticles with phosphoric acid methacrylates. For example, the nanoparticles may be reacted with hema phoshphate (HEMA-P) or bis hema phosphate (BisHEMA-P). The reacted product would be similar to the reaction product of zinc phosphate cements and the reactive monomer would also be available for further reaction with other methacrylate monomers. Other similar phosphoric acid methacrylates may be used for this purpose. Examples are: hema phosphate (HEMA-P), glycerophosphoric acid dimethacrylate (GPDM), pentaerythritol trimethacrylate phosphate (PENTA-P), 10-Methacryloxydecyl dihydrogen phosphate (10-MDP) and other phosphoric acid methacrylates typically used in dental adhesives.

A preferred method to prepare stable transparent zinc oxide nanoparticles is to react the particles with polyacrylic acid. In this case, the acid acrylate monomer is already polymerized, and the acid function of the polymer is available for reaction. The reaction is the same as for polycarboxylate cements. An advantage of this reaction is that polyacrylic acid is water soluble and results in a transparent precipitate that may be dried and milled as an additive. Moreover, the zinc oxide nanoparticles are sealed within the polyacrylic acid matrix and is stabilized over time. The ultimate product is essentially a polycarboxylate cement. This preferred method results in a permanently stable product that does not grow in particle size. The other methods, using coupling monomers are disfavored because they may still aggregate in paste form, becoming more opaque over time.

Example 2 describes the synthesis of the nanoparticles using polyacrylic acid to stabilize the nanoparticles.

### Example 2. Synthesis of ZnO Nanaoparticles Stabilized with Polycarboxylic Acid

1. Dissolve zinc acetate in distilled water. Mix at about 68° C. until dissolved.
2. Add NaOH solution and stir for at least 1 hr.
3. Dissolve polyacrylic acid in. distilled water. Take half of this solution and add it to the dissolved zinc acetate slowly to adjust to pH 4. Stir for at least an additional 2 hours and add remainder of the polyacrylic acid solution. Continue stirring at least 8 hours. A translucent paste will form.
4. Decant and collect precipitate. Dry overnight at about 80° C.
5. Grind to a powder with a mortar/pestle and mill for at least 8 hours with a grinding media. Screen the resultant powder through a mesh nylon screen. The powder may be used in high concentrations in restorative products with high translucency.

By incorporating stabilized zinc oxide nanoparticles in dental compositions provides several major benefits. Use to stabilized zinc oxide nanoparticles in dental compositions allows for a dental composition to incorporate a larger concentration of zinc oxide particles without sacrificing translucency. For example, when using bulk zinc oxide, a typical dental composition cannot exceed 0.8% zinc oxide in order to maintain translucency.

In contrast, use of stabilized zinc oxide nanoparticles allows larger concentrations of zinc oxide to be incorporated into a dental composition. For example, a dental composition containing 2-5% of stabilized zinc oxide nanoparticles maintains or has better translucency than dental compositions containing 0.1-0.8% of bulk zinc oxide.

With the increased concentration of stabilized nanoparticle zinc oxide present in the dental composition, this dental composition exhibits superior anti-microbial activity (see Table 1 below).

In order for transparent zinc oxide nanoparticles to perform effectively in esthetic dental materials, it must show similar antimicrobial activity as existing restoratives, adhesives, cements and bases. The AATCC Method-100 was chosen to show this. It is a surface contact method that does not use agar as a medium, which can complex with zinc ions. Esthetics was measured by opacity data over time. The control was the material without zinc oxide as a negative control.

The preferred form of stabilized zinc oxide nanoparticles was selected for evaluation. This was reacted zinc oxide nanoparticles locked into a polymerized resin matrix, polyacrylic acid, (ZnO:PAC nanoparticles). The reacted nanoparticles are not true zinc oxide nanoparticles but are the same as the reaction product of polycarboxylate cements. This product still has antimicrobial activity but is milder than pure zinc oxide nanoparticles.

A comparison of antimicrobial activity between bulk zinc oxide typically used in dental compositions with stabilized nanoparticle zinc oxide is presented in Table 1 below.

Example 3 is an embodiment where transparent nanoparticle zinc oxide was stabilized and then formulated into a dental adhesive. Example 4 is another embodiment where transparent nanoparticle zinc oxide was stabilized and then formulated into a dental adhesive.

Example 3. Dental Adhesive Containing
Transparent Zinc Oxide Nanoparticles

| Chemical | Weight/weight % |
|---|---|
| Resin | 25-40 |
| HEMA | 25-30 |
| Photoinitiator | 0.1-0.3 |
| Filler | 30-50 |
| Transparent Zinc Oxide Nanoparticles | 2.0-5.0 |

Example 4. Dental Restorative Containing
Transparent Zinc Oxide Nanoparticles

| Chemical | Weight/weight % |
|---|---|
| Ethyl 4-dimethylaminobenzoate | 0.05-0.15 |
| Photo Initiator | 0.05-0.15 |
| Resin Blend | 20-35 |
| 2-(2′ hydroxy-5′-octylphenyl) benzotriazole | 0.4-0.5 |
| Butylated hydroxytoluene | 0.008-0.01 |
| Filler | 75-85 |
| Fumed Silica blend | 0.5-1.5 |
| Transparent Zinc Oxide Nano particles | 2.0-5.0 |

Example 3 and Example 4 were tested for antimicrobial activity and translucency. A commercially available dental adhesive available under the tradename, Tenure S® sold by Den-Mat Holdings, LLC, was used as the control group for the dental adhesive. A commercially available dental restorative available under the tradename, Nuance®, was used as the control group for the dental restorative. Both Tenure S® and Nuance® were selected as the control groups because neither incorporate nanoparticle zinc oxide in the formulation. The results of these tests are displayed in Table 1 below. As described above, antimicrobial activity was measured according to Method AATCC-100. The opacity was also measured according to DenMat Test Specification, P-011: Translucency Check. The translucency is needed to assure that the formulation meets the esthetic requirements of a restorative material.

TABLE 1
Zinc Oxide Nanoparticles Test Data

AATCC-100 TEST

| | | Reduction | Reduction |
|---|---|---|---|
| MATERIAL | OPACITY | e. coli | Strep mutans |
| Example 3 | Passes | 100% | 100% |
| Tenure S | Passes | 3.6 × 10\hat{ }5 | 7.2 × 10\hat{ }5 |
| | | cfu/ml | cfu/ml |
| Example 4 | Passes | 100% | 100% |
| Nuance | Passes | 0% | 0% |

As Table 1 depicts, both Example 3 and 4 have 100% reduction in E. coli and Strep mutans. Whereas Tenure S® and Nuance® fail to achieve complete reduction in both E. coli and Strep mutans. Equally important is that Example 3 and Example 4 both pass the opacity test despite the high concentration of nanoparticle zinc oxide. If Example 3 and Example 4 had the same concentration of zinc oxide, but in bulk form instead of nanoparticle form, both would fail the opacity test due to having an opaque appearance.

The foregoing description of the invention with the accompanying examples is not intended to be limiting. It is contemplated that other embodiments may be made without departing from the spirit or scope of the invention as set forth in the appended claims

What is claimed is:

1. A method of producing optically stable transparent zinc oxide comprising the steps of:
dissolve zinc acetate in distilled water and mix at 68° C. until dissolved to form a zinc acetate solution;
add a base to said zinc acetate solution and stir for at least 1 hour;
dissolve an acid in distilled water to form a dilute acid solution;
slowly add said dilute acid solution to said zinc acetate solution until said zinc acetate solution reaches pH 4 and then stir for at least 2 hours and then add remainder of said acid solution to said zinc acetate solution and sit for at least 8 hours to form a translucent paste;
decant and collect said translucent paste and dry at least 8 hours;
grid said dried translucent paste using grinding media to form a powder; and
screen said powder through a mesh screen.

2. The method of claim 1 wherein said powder is optically sable nanoparticle zinc oxide with a particle size in the range of 1-15 nm.

3. The method of claim 1 wherein said base is NaOH.

4. The method of claim 3 wherein said NaOH has a molar concentration of 2M.

5. The method of claim 1 wherein said acid is polyacrylic acid.
